(19) United States
(12) Patent Application Publication
(10) Pub. No.: US 2010/0305207 A1
(43) Pub. Date: Dec. 2, 2010

(54) GLYCOGEN PHOSPHORYLASE INHIBITOR COMPOUND AND PHARMACEUTICAL COMPOSITION THEREOF

(76) Inventors: Pierette Banker, Durham, NC (US); Eric Eugene Boros, Durham, NC (US); Scott Howard Dickerson, Durham, NC (US); Istvan Kaldor, Durham, NC (US); Cecilia S. Koble, Durham, NC (US); Michael Tolar Martin, Durham, NC (US); Steven Meagher Sparks, Durham, NC (US); Stephen Andrew Thomson, Durham, NC (US)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(21) Appl. No.: 12/677,846

(22) PCT Filed: Sep. 25, 2008
(86) PCT No.: PCT/US08/77624

§ 371 Date: Mar. 12, 2010

Related U.S. Application Data
(60) Provisional application No. 60/975,862, filed on Sep. 28, 2007.

(51) Int. Cl. A61K 31/197; C07C 275/42; A61P 3/10; A61P 3/04; A61P 3/06; A61P 9/00

(52) U.S. Cl. 514/563; 562/439

(57) ABSTRACT

This invention relates to a novel compound which is a glycogen phosphorylase inhibitor and its use in the treatment of diabetes and other conditions associated therewith. The invention further relates to a pharmaceutical composition containing the compound and to processes for preparing the compound and pharmaceutical composition.

### FIELD OF THE INVENTION

The present invention relates to a glycogen phosphorylase inhibitor compound, a pharmaceutical composition of the compound, the use of the compound or pharmaceutical composition containing it in the treatment of diabetes, conditions associated with diabetes, and/or tissue ischemia, including myocardial ischemia, and a process for making the compound.

### BACKGROUND OF THE INVENTION

Treatment of diabetes remains a health concern in much of the world. Orally ingested drugs having minimal undesirable side effects are desired over the self-injection of insulin. There is a continuing need for drugs that are better, having fewer side effects, longer acting, or act via different mechanisms.

A number of drugs are available for the treatment of diabetes. These include injected insulin and drugs such as sulfonylureas, glipizide, tobutamide, acetohexamide, tolazimide, biguanides, and metformin (glucophage) which are ingested orally. Insulin self-injection is required in diabetic patients in which orally ingested drugs are not effective. Patients having Type 1 diabetes (also referred to as insulin dependent diabetes mellitus) are usually treated by self-injecting insulin. Patients suffering from Type 2 diabetes (also referred to as non-insulin dependent diabetes mellitus) are usually treated with a combination of diet, exercise, and an oral agent. When oral agents fail, insulin may be prescribed. When diabetic drugs are taken orally, usually multiple daily doses are often required.

Determination of the proper dosage of insulin requires frequent testing of the level of sugar in a patient's urine and/or blood. The administration of an excess dose of insulin generally causes hypoglycemia which has symptoms ranging from mild abnormalities in blood glucose to coma, or even death. Orally ingested drugs are, likewise, not without undesirable side effects. For example, such drugs can be ineffective in some patients and cause gastrointestinal disturbances or impair proper liver function in other individuals. There is always a need for improved drugs having fewer side effects and/or ones that succeed where others fail.

In Type 2 or non-insulin dependent diabetes mellitus, hepatic glucose production is an important target. The liver is the major regulator of plasma glucose levels in the fasting state. The rate of hepatic glucose production in Type 2 patients is typically significantly elevated when compared to non-diabetic individuals. For Type 2 diabetics, in the fed or postprandial state, the liver has a proportionately smaller role in the total plasma glucose supply, and hepatic glucose production is abnormally high.

The liver produces glucose by glycogenolysis (breakdown of the glucose polymer glycogen) and gluconeogenesis (synthesis of glucose from 2- and 3-carbon precursors). Glycogenolysis, therefore, is an important target for interruption of hepatic glucose production. There is some evidence to suggest that glycogenolysis may contribute to the inappropriate hepatic glucose output in Type 2 diabetic patients. Individuals having liver glycogen storage diseases such as Hers' disease or glycogen phosphorylase deficiency often display episodic hypoglycemia. Further, in normal post-absorptive humans up to about 75% of hepatic glucose production is estimated to result from glycogenolysis.

Glycogenolysis is carried out in liver, muscle, and brain by tissue-specific isoforms of the enzyme glycogen phosphorylase. This enzyme cleaves the glycogen macromolecule to release glucose-1-phosphate and a shortened glycogen macromolecule.

Glycogen phosphorylase inhibitors include glucose and its analogs, caffeine and other purine analogs, cyclic amines with various substitutents, acyl ureas, and indole-like compounds. These compounds and glycogen phosphorylase inhibitors, in general, have been postulated to be of potential use in the treatment of Type 2 diabetes by decreasing hepatic glucose production and lowering glycemia. Furthermore, it is believed desirable that a glycogen phosphorylase inhibitor be sensitive to glucose concentrations in blood.

Accordingly, what is desired is a new compound and pharmaceutical composition containing it for the treatment of diabetes and/or conditions associated with diabetes.

### SUMMARY OF THE INVENTION

The present invention provides a compound of Formula I,

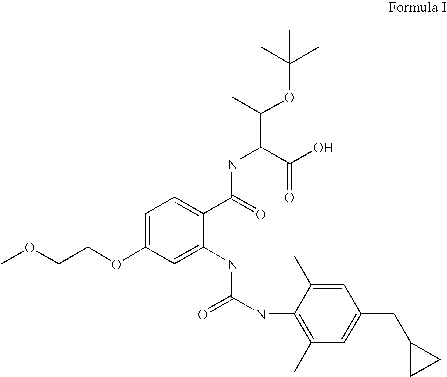

salt, solvate, or physiological functional derivative thereof.

There is also provided a pharmaceutical composition comprising a compound of Formula I, salt, solvate, or physiologically functional derivative thereof.

Further, there is provided a pharmaceutical composition comprising a compound of Formula I, salt, solvate, or physiologically functional derivative thereof and one or more excipients.

There is still further provided a method of treatment comprising administering to a mammal, particularly a human, a pharmaceutical composition comprising a compound of Formula I, pharmaceutically acceptable salt, solvate, or physiologically functional derivative thereof and at least one excipient, wherein said treatment is for a disease or condition selected from the group consisting of diabetes, conditions associated with diabetes, and tissue ischemia, including myocardial ischemia.

Additionally, there is provided a compound of Formula I, salt, solvate, or physiologically functional derivative thereof for use as an active therapeutic substance (in therapy). And, there is also provided a compound of Formula I, salt, solvate, or physiologically functional derivative thereof for use in the treatment of diabetes, conditions associated with diabetes, and/or tissue ischemia, including myocardial ischemia in a mammal, especially a human.

A process for preparing a compound of Formula I, salt, solvate, or physiologically functional derivative thereof is also provided.

### DETAILED DESCRIPTION OF THE INVENTION

The activity of glycogen phosphorylase in muscle tissue is important for the generation of glucose and subsequently energy demand. Inhibition of muscle glycogen phosphorylase at the time of exercise may lead to muscle weakness and muscle tissue damage. Therefore, it may be desirable to have the compound of the present invention which shows a greater effect on glycogen phosphorylase in the liver as compared to the muscle when given orally to mammals. The compound of the present invention shows a strong effect on liver glycogen content with little effect on muscle glycogen content and function after an oral dose. Consequently, the compound of the present invention could exhibit potent in vivo activity, have acceptable solubility and bioavailability properties, as well as having an improved safety/toxicity profile in view of its selectivity for liver tissue.

The present invention provides a compound of Formula I

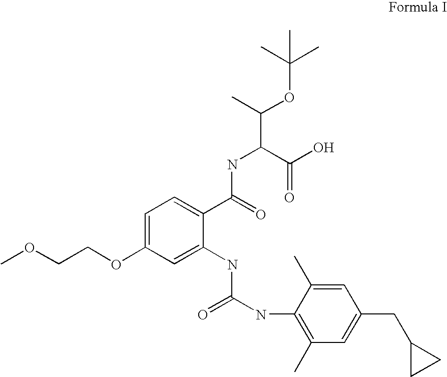

salt, solvate, or physiological functional derivative thereof. The chemical name for a compound of Formula I is N-[(2-[({[4-(cyclopropylmethyl)-2,6-dimethylphenyl]amino}carbonyl)amino]-4-{[2-(methyloxy)ethyl]oxy}phenyl)carbonyl]-O-(1,1-dimethylethyl)threonine.

The compound of Formula I or a salt, solvate, or physiologically functional derivative thereof may exist in stereoisomeric forms (e.g., it contains one or more asymmetric carbon atoms). The individual stereoisomers (enantiomers and diastereomers) and mixtures of these are included within the scope of the present invention. The invention also covers the individual isomers of the compound (salt, solvate, or physiologically functional derivative) represented by Formula I as mixtures with isomers thereof in which one or more chiral centers are inverted. Likewise, it is understood that a compound (salt, solvate, or physiologically functional derivative) of Formula I may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are individual isomers of the compound represented by Formula I, as well as any wholly or partially equilibrated mixtures thereof. The present invention also includes the individual isomers of the compound, salt, solvate, or derivative represented by the formula as well as mixtures with isomers thereof in which one or more chiral centers are inverted. It is to be understood that the present invention includes all combinations and subsets of the particular groups defined hereinabove.

The preferred stereochemistry of the compound is shown in Formula IA below:

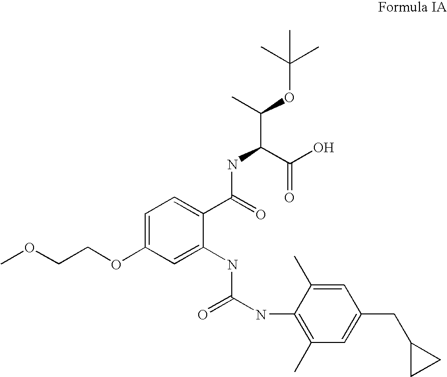

It will be appreciated by those skilled in the art that the compound of the present invention may also be utilized in the form of a pharmaceutically acceptable salt, solvate, or physiologically functional derivative thereof.

Typically, but not absolutely, the salts of the present invention are pharmaceutically acceptable salts. Salts encompassed within the term “pharmaceutically acceptable salts” refer to non-toxic salts of the compound of the invention. Salts of the compound of the present invention may include conventional salts formed from pharmaceutically acceptable inorganic or organic bases. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine, and procaine salts.

As used herein, the term “solvate” refers to a complex of stoichiometry formed by a solute (in this invention, a compound of Formula I, salt, or physiologically functional derivative thereof) and a solvent. Such solvents, for the purpose of the invention, may not interfere with the biological activity of the solute. Non-limiting examples of suitable solvents include, but are not limited to water, methanol, ethanol, and acetic acid. Preferably the solvent used is a pharmaceutically acceptable solvent. Most preferably the solvent used is water and the solvate is a hydrate.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry and Drug Discovery, 5^{th} Edition, Volume 1: Principles and Practice, which is incorporated herein by reference to the extent that it teaches physiologically functional derivatives.

Processes for preparing pharmaceutically acceptable salts, solvates, and physiologically functional derivatives of the compound of Formula I are generally known in the art. See, for example, Burger's Medicinal Chemistry and Drug Discovery, 5^{th} Edition, Volume 1: Principles and Practice.

The compound (salt, solvate, or physiologically functional derivative) of Formula I may be conveniently prepared by the process outlined below. The order of the foregoing steps is not critical to the practice of the invention and the process may be practiced by performing the steps in any suitable order based on the knowledge of those skilled in the art. In addition some of the steps described may be combined without the isolation of all intermediate compounds.

One general method of the synthesis of the compound of Formula I is outlined in Scheme 1 below. The starting 4-fluoro-2-nitrobenzoic acid (2) can be converted to the methoxyethyl ester under standard conditions, such as treatment with 2-bromoethyl methyl ether and a base such as potassium carbonate in a polar solvent such as DMF or NMP. The fluoro group can be displaced with 2-methoxyethanol under basic conditions such as potassium carbonate in DMF or NMP. The ester can then be removed under basic conditions such as lithium hydroxide or sodium hydroxide in solvents which include tetrahydrofuran (THF) and/or methanol (MeOH) and/or water and/or 1,4-dioxane to give Intermediate 3.

Intermediate 5 is formed by mixing intermediate 3 with methyl O-(1,1-dimethylethyl)-L-threoninate (4) or its hydrochloride salt under standard coupling conditions. These conditions include, but are not limited to, the use of EDC (1-ethyl-3-(3-dimethylaminopropyl)carbodiimide hydrochloride), PyBop (benzotriazole-1-yl-oxy-tris-pyrrolidino-phosphonium hexafluorophosphate), PyBrOP (bromo-tris-pyrrolidino-phosphonium hexafluorophosphate), HOBT (N-hydroxybenzotriaole), HOAT (N-hydroxy-9-azabenzotriazole), or DIC (N,N′-diisopropylcarbodiimide), or HATU (2-(1H-9-azabenzotriazole-1-yl)-1,1,3,3-tetramethyluronium hexafluorophosphate) and DIEA (N,N-diisopropylethylamine) or triethylamine at room temperature. Solvents that can be used include DMSO, NMP, or preferably DMF. In addition, combining 3 and 4 in ethyl acetate in the presence of 1-propanephosphonic acid cyclic anhydride and an organic base such as DIEA or triethylamine will yield intermediate 5. In a preferred method, 3 is converted into the corresponding acid chloride under standard conditions such as treatment with oxalyl chloride in a solvent such as dichloromethane, in the presence of a catalytic amount of DMF. Reaction of the resulting acid chloride with methyl O-(1,1-dimethylethyl)-L-threoninate (4) or its hydrochloride salt in the presence of an organic base such as DIEA or triethylamine in a solvent such as acetonitrile yields intermediate 5.

Reduction of the nitro group of 5 under standard conditions such as, but not limited to, treatment with palladium on carbon under a hydrogen atmosphere in a solvent such as ethyl acetate or methanol yields intermediate 6.

Intermediate 8 is formed by mixing intermediate 6 with the isocyanate, intermediate 7 (method of synthesis outlined below, see Scheme 3) and diisopropylethylamine (DIEA) or triethylamine, in a solvent such as DMF. Preferably intermediates 6 and 7 are combined in pyridine to give intermediate 8.

The final product is formed by cleavage of the ester of intermediate 8 under basic conditions such as lithium hydroxide or sodium hydroxide in solvents which include tetrahydrofuran (THF) and/or methanol (MeOH) and/or water and/or 1,4-dioxane.

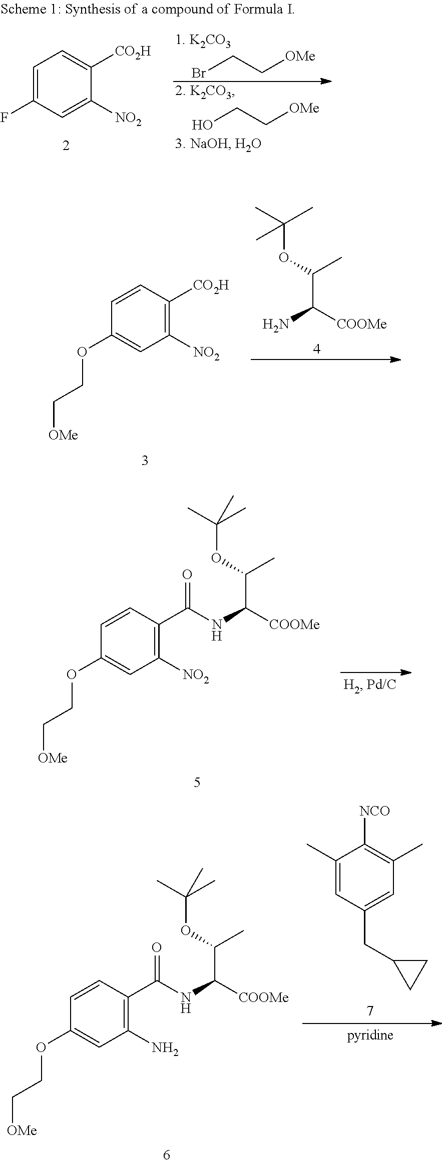

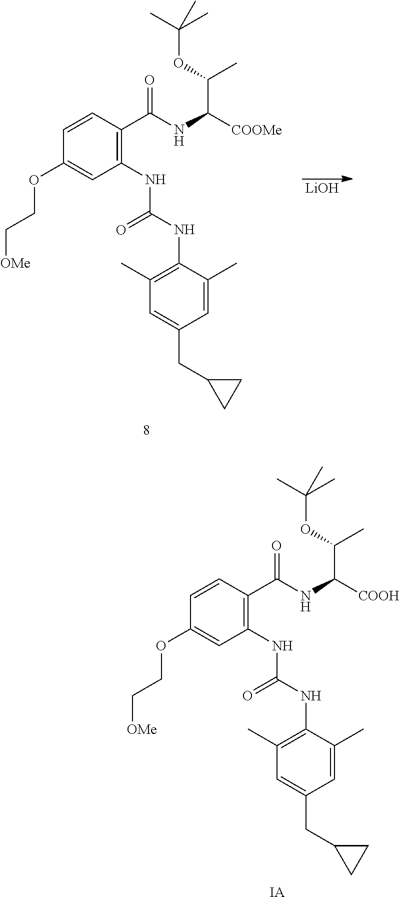

Alternatively, to synthesize other isomers or racemates of Formula I, a different isomer of (4) can be substituted. An alternate route to intermediate 3 is shown in Scheme 2 below. Intermediate 9 is converted to the phenol, intermediate 10, by treatment with sodium nitrite and hydrochloric acid. Intermediate 3 is then prepared by reaction of intermediate 10 with 2-bromoethyl methyl ether under basic conditions such as potassium carbonate in DMF, followed by treatment with lithium hydroxide or sodium hydroxide in solvents which include tetrahydrofuran (THF) and/or methanol (MeOH) and/or water and/or 1,4-dioxane.

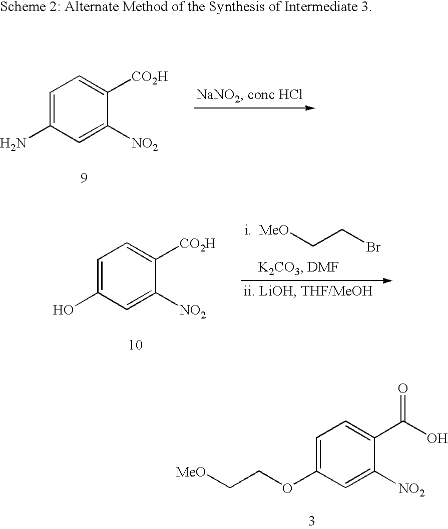

One general method of the synthesis of intermediate 7 is outlined in Scheme 3 below. Treatment of the commercially available 4-bromo-2,6-dimethylaniline (11) with Vilsmeier reagent ((chloromethylene)dimethyliminium chloride, or generation in situ from DMF and oxalyl chloride) in a solvent such as dichloromethane will give intermediate 12. Reaction of intermediate 12 with a strong base such as n-butyl lithium at low temperature in a solvent such as THF followed by treatment with cyclopropanecarbaldehyde yields intermediate 13.

Intermediate 14 is formed by treatment of intermediate 13 with lithium hydroxide in refluxing ethanolamine followed by treatment with lithium hydroxide in isopropyl alcohol and water.

Reduction of intermediate 14 to give intermediate 15 can be carried out by treatment of intermediate 14 with triethylsilane and trifluoroacetic acid. In an alternative method the benzylic hydroxyl of intermediate 13 can be reduced by treatment with borane THF complex and boron trifluoride diethyl etherate in THF. The resulting methylene compound is then treated with lithium hydroxide, hydrazine, in ethanol and water with heating to give intermediate 15.

Intermediate 7 is then obtained by treatment of intermediate 15 with phosgene or triphosgene and a base such as DIEA in a solvent such as dichloromethane.

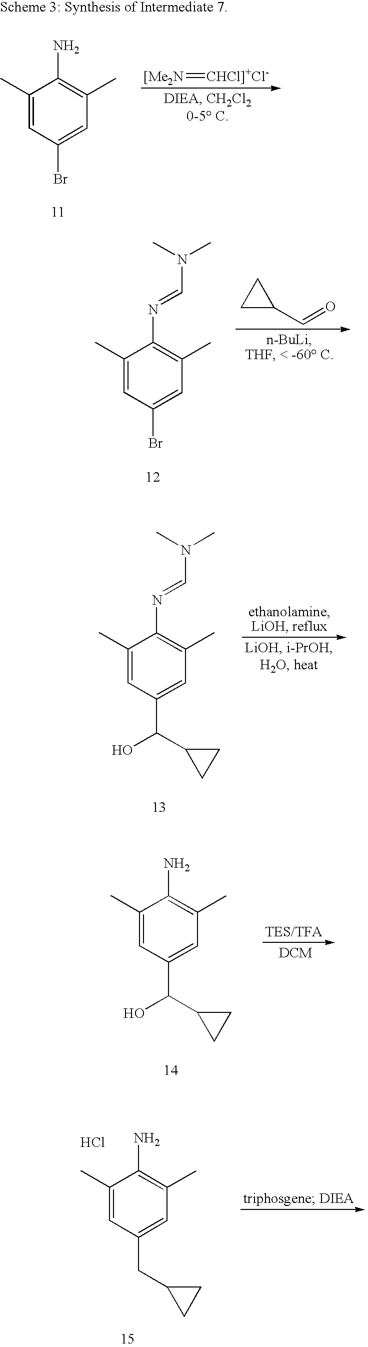

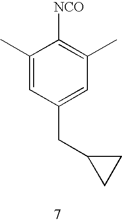

The invention further provides a pharmaceutical composition (also referred to as pharmaceutical formulation) comprising a compound of Formula I, salt, solvate, or physiologically functional derivative thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

In accordance with another aspect of the invention there is provided a process for the preparation of a pharmaceutical composition comprising mixing (or admixing) a compound of Formula I, salt, solvate, or physiologically functional derivative thereof with at least one excipient.

Pharmaceutical compositions may be in unit dose form containing a predetermined amount of active ingredient per unit dose. Such a unit may contain a therapeutically effective dose of the compound of Formula I, salt, solvate, or physiologically functional derivative thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound (salt, solvate, or derivative) of the invention or pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound (salt, solvate, or derivative) of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound (salt, solvate, or derivative) of the invention in a suitably flavoured aqueous solution, while elixirs are prepared through the use of a non-toxic alcoholic vehicle. Suspensions can be formulated by dispersing the compound (salt, solvate, or derivative) of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject.

Prophylaxis (or prevention or delay of disease onset) is typically accomplished by administering a drug in the same or similar manner as one would to a patient with the developed disease or condition.

The present invention provides a method of treatment in a mammal, especially a human, suffering from diabetes or a related condition such as obesity, syndrome X, insulin resistance, diabetic nephropathy, diabetic neuropathy, diabetic retinopathy, hyperglycemia, hypercholesterolemia, hyperinsulinemia, hyperlipidemia, cardiovascular disease, stroke, atherosclerosis, lipoprotein disorders, hypertension, tissue ischemia, myocardial ischemia, and depression. Such treatment comprises the step of administering a therapeutically effective amount of a compound of Formula I, salt, solvate, or physiologically functional derivative thereof to said mammal, particularly a human. Treatment can also comprise the step of administering a therapeutically effective amount of a pharmaceutical composition containing a compound of Formula I, salt, solvate, or physiologically functional derivative thereof to said mammal, particularly a human.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula I, as well as salts, solvates, and physiologically functional derivatives thereof, may be administered as the raw chemical. Additionally, the active ingredient may be presented as a pharmaceutical composition.

While it is possible that, for use in therapy, a therapeutically effective amount of a compound of Formula I (salt, solvate, or physiologically functional derivative thereof) may be administered as the raw chemical, it is typically presented as the active ingredient of a pharmaceutical composition or formulation.

The precise therapeutically effective amount of a compound (salt, solvate, or physiologically functional derivative) of the invention will depend on a number of factors, including, but not limited to, the age and weight of the subject (patient) being treated, the precise disorder requiring treatment and its severity, the nature of the pharmaceutical formulation/composition, and route of administration, and will ultimately be at the discretion of the attending physician or veterinarian. Typically, a compound of Formula I (salt, solvate, or physiologically functional derivative thereof) will be given for the treatment in the range of about 0.1 to 100 mg/kg body weight of recipient (patient, mammal) per day and more usually in the range of 0.1 to 10 mg/kg body weight per day. Acceptable daily dosages may be from about 1 to about 1000 mg/day, and preferably from about 1 to about 100 mg/day. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of Formula I per se. Similar dosages should be appropriate for treatment (including prophylaxis) of the other diseases/conditions referred herein for treatment. In general, determination of appropriate dosing can be readily arrived at by one skilled in medicine or the pharmacy art.

Additionally, the present invention comprises a compound of Formula I, salt, solvate, or physiological functional derivative thereof, or a pharmaceutical composition thereof with at least one other anti-diabetic drug. Such anti-diabetic drugs can include, for example, injected insulin and drugs such as sulfonylureas, thiazolidinediones, glipizide, glimepiride, tobutamide, acetohexamide, tolazimide, biguanides, rosiglitazone, metformin (glucophage), sitagliptin (Januvia) salts or combinations thereof, and the like, which are ingested orally. When a compound of the invention is employed in combination with another anti-diabetic drug, it is to be appreciated by those skilled in the art that the dose of each compound or drug of the combination may differ from that when the drug or compound is used alone. Appropriate doses will be readily appreciated and determined by those skilled in the art. The appropriate dose of the compound of Formula I (salt, solvate, physiologically functional derivative thereof) and the other therapeutically active agent(s) and the relative timings of administration will be selected in order to achieve the desired combined therapeutic effect, and are with the expertise and discretion of the attending doctor or clinician.

### EXPERIMENTAL

The following examples are intended for illustration only and are not intended to limit the scope of the invention in any way, the invention being defined by the claims. Unless otherwise noted, reagents are commercially available or are prepared according to procedures in the literature.

### Example 1

### Preparation of the Compound of Formula IA

### N-[(2-[({[4-(Cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threonine

### Step 1. 2-(Methyloxy)ethyl 4-fluoro-2-nitrobenzoate

4-Fluoro-2-nitrobenzoic acid (15 g, 81 mmol) was dissolved in DMF (80 mL) and 2-bromoethyl methyl ether (12.39 g, 89.1 mmol) was added followed by potassium carbonate (16.8 g, 121.5 mmol). The solution was heated to 85° C. for 5 h, cooled, and poured onto water and ethyl acetate. The organic layer was separated and washed with brine, dried (MgSO₄), and concentrated to give 21.7 g of a yellow oil.

¹H NMR (400 MHz, DMSO-D6) δ ppm 8.07 (dd, J=8.3, 2.4 Hz, 1H), 7.96 (dd, J=8.4, 2.4 Hz, 1H), 7.72 (dt, J=8.4, 2.4 Hz, 1H), 4.35 (m, 2H), 3.57 (m, 2H), 3.25 (s, 3H).

### Step 2. 2-(Methyloxy)ethyl 4-{[2-(methyloxy)ethyl]oxy}-2-nitrobenzoate

2-(Methyloxy)ethyl 4-fluoro-2-nitrobenzoate (5 g, 20.56 mmol) was dissolved in DMF (40 mL) and 2-methoxyethanol (4.7 g, 61.68 mmol) and potassium carbonate (5.7 g, 41.12 mmol) were added. The solution was heated to 85° C. for 15 h and cooled. The mixture was poured onto water and ethyl acetate and the organic layer was washed with water and brine, dried (MgSO₄), and concentrated to give 4.83 g (78%) of desired product as a gold oil.

¹H NMR (400 MHz, DMSO-D6) δ ppm 7.8 (d, J=8.8 Hz, 1H), 7.6 (d, J=2.4 Hz, 1H), 7.3 (dd, J=8.7, 2.6 Hz, 1H), 4.3 (m, 2H), 4.2 (m, 2H), 3.7 (m, 2H), 3.6 (m, 2H), 3.3 (s, 3H), 3.2 (s, 3H).

### Step 3. 4-{[2-(Methyloxy)ethyl]oxy}-2-nitrobenzoic Acid

2-(Methyloxy)ethyl 4-{[2-(methyloxy)ethyl]oxy}-2-nitrobenzoate (4.83 g, 16.13 mmol) was dissolved in 1:1 THF/MeOH (80 mL) and 2 M LiOH (80 mL) was added. After 4 h the reaction was concentrated to ˜80 mL and 5 M HCl (34 mL) was added. The solution was extracted with ethyl acetate and the extracts were dried (MgSO₄) and concentrated to afford 3.70 g (95%) of product as a pale yellow solid.

¹H NMR (400 MHz, DMSO-D6) δ ppm 3.3 (s, 3H) 3.6 (m, 2H) 4.2 (m, 2H) 7.3 (dd, J=8.7, 2.6 Hz, 1H) 7.5 (d, J=2.4 Hz, 1H) 7.8 (d, J=8.8 Hz, 1H).

### Step 4. Methyl O-(1,1-dimethylethyl)-N-[(4-{[2-(methyloxy)ethyl]oxy}-2-nitrophenyl)carbonyl]-L-threoninate

4-{[2-(Methyloxy)ethyl]oxy}-2-nitrobenzoic acid (3.7 g, 15.4 mmol) and methyl O-(1,1-dimethylethyl)-L-threoninate hydrochloride (3.8 g, 16.8 mmol) were dissolved in ethyl acetate (50 mL) and triethylamine (4.66 g, 46.0 mmol) was added. The reaction was heated to 70° C. and 1-propanephosphonic acid cyclic anhydride (19.5 g of a 50% solution in ethyl acetate, 30.6 mmol) was added dropwise. After 10 h the solution was cooled to RT, washed with water and brine, dried (MgSO₄), and concentrated to give 6.84 g (108%) of product as a viscous oil.

¹H NMR (400 MHz, DMSO-D6) δ ppm 1.1 (m, 9H) 1.1 (d, J=6.1 Hz, 3H) 3.3 (s, 3H) 3.6 (s, 3H) 3.7 (m, 2H) 4.2 (m, 3H) 4.5 (dd, J=8.8, 3.2 Hz, 1H) 7.3 (dd, J=8.5, 2.7 Hz, 1H) 7.5 (m, 2H) 8.7 (d, J=8.8 Hz, 1H).

### Step 5. Methyl N-[(2-amino-4-{[2-(methyloxy)ethyl]oxy}phenyl)carbonyl]-O-(1,1-dimethylethyl)-L-threoninate

Methyl O-(1,1-dimethylethyl)-N-[(4-{[2-(methyloxy)ethyl]oxy}-2-nitrophenyl)carbonyl]-L-threoninate (6.32 g, 15.3 mmol) was dissolved in ethyl acetate (100 mL) and 10% Pd/C (600 mg) was added. The reaction was placed on a Parr hydrogenator under 60 psi of H₂ and the mixture was shaken. The reactor was recharged to 60 psi of H₂ twice over a 48 h period. The reaction was removed from the apparatus, filtered through celite, and concentrated. The residue was purified by SiO₂ chromatography (80 g SiO₂, 10-50% ethyl acetate/hexanes) to afford 4.83 g (82%) of product as a viscous gold oil.

¹H NMR (400 MHz, DMSO-D6) δ ppm 1.1 (s, 9H) 1.1 (d, J=6.1 Hz, 3H) 3.3 (s, 3H) 3.6 (m, 5H) 4.0 (m, 2H) 4.1 (dd, J=6.2, 3.3 Hz, 1H) 4.4 (dd, J=8.3, 3.2 Hz, 1H) 6.1 (dd, J=8.8, 2.4 Hz, 1H) 6.2 (d, J=2.4 Hz, 1H) 6.5 (s, 2H) 7.3 (d, J=8.5 Hz, 1H) 7.4 (d, J=8.8 Hz, 1H).

### Step 6. (4-Amino-3,5-dimethylphenyl)(cyclopropyl)methanol

A 10 L jacketed laboratory reactor, equipped with a mechanical stirrer, was charged with dichloromethane (6 L) and DMF (201 g, 2.75 mol). The jacket was cooled to 15° C. and oxalyl chloride (348.8 g, 2.75 mol) was added over ca. 20 min. After stirring an additional 5 min, 4-bromo-2,6-dimethylaniline (500 g, 2.50 mol) in dichloromethane (1 L) was added. The reaction mixture was stirred for 30 min and then the dichloromethane layer was washed with 2N NaOH (3 L), water (2 L) and 50% brine (1 L). The dichloromethane layer was then distilled to a volume of ca. 1 L. THF (4 L) was added, and the mixture was then distilled to a volume of 2 L, THF (4 L) was added again and the mixture was distilled to 2 L, and a final 5 L of THF was added. The resulting solution was cooled to ca. −70° C. and n-butyl lithium (2.5 M in hexanes, 1.25 L. 3.12 mol) was added over ca. 40 min, in which the internal temperature was maintain at <−50° C. After the addition was complete, the reaction was cooled to −60° C. and cyclopropanecarbaldehyde (221.45 g, 3.12 mol) was added at such a rate to maintain the internal temperature <−48° C. The mixture was then allowed to warm to RT and water (2 L) was added. The mixture was distilled to a volume of ca. 2 L, then lithium hydroxide monohydrate (315 g, 7.50 mol) in water (2 L) and ethanolamine (460 g, 7.5 mol) was added. The mixture was brought to reflux and isopropanol (1 L) was added. After refluxing for ca. 18 h the reaction was cooled to RT, and the pH was adjusted to ca. 10 with concentrated HCl. The mixture was extracted with ethyl acetate (2×2 L, 1×1 L) and the combined organics were washed with water (1 L). The resulting solution was concentrated to ca. 1 L, heated to reflux and then cooled to 0° C. The resulting solids were collected by filtration, washed with ethyl acetate and hexanes, and dried under vacuum at ca. 45° C. to give 254.4 g (53%) of the product. ¹H NMR (400 MHz, DMSO-D6) δ ppm: 6.80 (s, 2H), 4.75 (brs, 1H), 4.38 (brs, 2H), 3.74 (d, J=7.2 Hz, 1H), 2.07 (s, 6H), 1.04-0.93 (m, 1H), 0.45-0.15 (m, 4H).

### Step 7. 4-(Cyclopropylmethyl)-2,6-dimethylaniline hydrochloride

To (4-amino-3,5-dimethylphenyl)(cyclopropyl)methanol (10.0 g, 52.3 mmol) in dichloromethane (100 mL) cooled to ca. 10° C. was added trifluoroacetic acid (7.15 g, 62.7 mmol) slowly. After stirring ca. 4 min triethylsilane (7.30 g, 62.7 mmol) was added dropwise over ca. 20 min. The reaction was stirred at ca. 10° C. for 40 min, and then concentrated under reduced pressure to ca. 20 mL and ethyl acetate (100 mL) was added. This was concentrated to ca. 50 mL, ethyl acetate was added (25 mL) and with stirring concentrated HCl (5 mL) was added. After stirring ca. 15 min the solids were collected by filtration, washed with ethyl acetate/hexanes (1:1) and dried under reduced pressure to give 10.1 g (91%) of the product. ¹H NMR (DMSO-d₆, 400 MHz) δ ppm: 9.15-9.75 (brs, 2H), 6.95 (s, 2H), 2.36 (d, J=6.8 Hz, 2H), 2.28 (s, 6H), 0.88 (m, 1H), 0.40 (m, 2H), 0.12 (m, 2H).

This reaction was repeated on 8 g of starting material and the resulting product (8.1 g) was combined with the material above.

### Step 8. 5-(Cyclopropylmethyl)-2-isocyanato-1,3-dimethylbenzene

A solution of 4-(cyclopropylmethyl)-2,6-dimethylaniline hydrochloride (11.1 g, 52.4 mmol) and diisopropylethylamine (10.0 mL, 57.7 mmol) dissolved in CH₂Cl₂ (50 mL) was added dropwise to a stirring solution of triphosgene (7.78 g, 26.2 mmol) in CH₂Cl₂ (100 mL). After addition was complete the solution was stirred for 45 min and then the solvent was concentrated off in a fume hood (Caution: un-reacted phosgene maybe present). The resulting residue was dissolved in heptanes, washed with water, dried (MgSO₄), and concentrated to afford 9.21 g (87%) of product as a cloudy viscous oil. ¹H NMR (400 MHz, DMSO-D6) 8 ppm: 0.1 (m, 2H) 0.4 (m, 2H) 0.9 (m, 1H) 2.2 (s, 6H) 2.4 (d, J=6.8 Hz, 2H) 7.0 (s, 2H).

### Step 9. Methyl N-[(2-[({[4-(cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threoninate

Methyl N-[(2-amino-4-{[2-(methyloxy)ethyl]oxy}phenyl)carbonyl]-O-(1,1-dimethylethyl)-L-threoninate (4.74 g, 12.39 mmol) was dissolved in pyridine (50 mL) and 5-(cyclopropylmethyl)-2-isocyanato-1,3-dimethylbenzene (3.74 g, 18.59 mmol) was added. The reaction was stirred for 7 h and then poured onto 1 M HCl layered with ethyl acetate. The organic layer was separated, washed with brine, dried (MgSO₄), and concentrated to a yellow oil. The oil was purified by SiO₂ chromatography (120 g SiO₂, 0-50% EA/hexanes) to afford 5.15 g (71%) of product as a colorless foam. ES MS m/z 584 (M+H).

### Step 10. N-[(2-[({[4-(Cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threonine

Methyl N-[(2-[({[4-(cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threoninate (5.1 g, 8.73 mmol) was dissolved in 1:1 THF/MeOH (40 mL) and 2 M LiOH (21.8 ml, 43.68 mmol) was added. After 3 h 1 M HCl (45 mL) and water (100 mL) was added and the mixture was extracted with EA. The extracts were dried (MgSO₄), combined with extracts from a second reaction starting from 0.88 g of starting material, and concentrated to give 5.97 g (102% overall) of product as a colorless foam.

¹H NMR (400 MHz, DMSO-D6) δ ppm: 0.2 (m, 2H) 0.4 (m, 2H) 0.9 (m, 1H) 1.1 (s, 12H) 2.1 (m, 6H) 2.4 (d, J=7.0 Hz, 2H) 3.3 (s, 3H) 3.6 (m, 2H) 4.1 (m, 2H) 4.2 (br s, 1H) 4.4 (br s, 1H) 6.6 (dd, J=8.7, 2.7 Hz, 1H) 6.9 (s, 2H) 7.7 (br s, 1H) 7.8 (br s. 1H) 8.0 (d, J=2.5 Hz, 1H) 8.7 (br s. 1H) 10.5 (br s, 1H) 12.8 (s, 1H). ES MS m/z 570 (M+H).

### Example 2

### Larger Scale Synthesis of Compound of Formula IA

### N-[(2-[({[4-(Cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threonine

### Step 1. 4-{[2-(Methyloxy)ethyl]oxy}-2-nitrobenzoic Acid

To 4-f| acid (200 g, 1.08 mol) in a jacketed laboratory reactor equipped with a mechanical stirrer, was added NMP (0.90 L) and potassium carbonate (520 g, 3.80 mol). The Reaction jacket was heated to 50° C. and 2-bromoethyl methyl ether (120 mL, 1.28 mol) was added over 10 min. The reaction jacket temperature was increased to ca. 129° C. and the reaction was stirred for 1 h. NMP (80 mL), 2-(methyloxy)ethanol (520 mL), and potassium carbonate (15 g) were added. The jacket temperature was increase to ca. 135° C. and the reaction was stirred for ca. 1 h, and then the jacket temperature was increased to 150° C. and the reaction was stirred for ca. 5 h. The mixture was cooled to RT and stirred for ca. 16 h. To this was added a mixture of 50% aqueous sodium hydroxide (235 mL) and water (0.77 L). After stirring 1 h the mixture was transferred to a bucket with water (0.50 L), acidified with 6 N HCl (ca. 2 L) and stirred for ca. 1.5 h. The resulting solids were collected by filtration and washed with water. Drying under reduced pressure at ca. 70° C. gave 160 g of the product. ¹H NMR (DMSO-d₆, 400 MHz) ppm: 13.53 (br s, 1H), 7.83 (d, J=8.5 Hz, 1H), 7.48 (d, J=2.5 Hz, 1H), 7.26 (dd, J=8.8, 6.2 Hz, 1H), 4.23 (m, 2H), 3.65 (m, 2H), 3.28 (s, 3H).

Additional smaller scale reactions of the above procedure were also performed to give additional material. Spectral properties were the same as above.

### Step 2. Methyl O-(1,1-dimethylethyl)-N-[(4-{[2-(methyloxy)ethyl]oxy}-2-nitrophenyl)carbonyl]-L-threoninate

To 4-{[2-(methyloxy)ethyl]oxy}-2-nitrobenzoic acid (205 g, 0.97 mol) dissolved in dichloromethane (2.5 L) and DMF (2.5 mL), in a jacketed laboratory reactor equipped with a mechanical stirrer, with the jacket temperature at ca. 27° C., was added oxalyl chloride (104 mL, 1.23 mol) dropwise over ca. 20 min. With the jacket temperature at 30° C. the reaction was stirred for ca. 2 h and then concentrated under reduced pressure. The residue was dissolved in acetonitrile (0.50 L), concentrated under reduced pressure, and re-dissolved in acetonitrile (0.30 L). This was then added dropwise to a cold (<10° C.) mixture of methyl O-(1,1-dimethylethyl)-L-threoninate hydrochloride (195 g, 0.86 mol), and DIEA (390 mL) dissolved in acetonitrile (1.5 L), in a jacketed laboratory reactor equipped with a mechanical stirrer. When the addition was complete the reaction was warmed to RT and diluted with methyl t-butyl ether (3 L) and washed with 1:1 brine:water (1.5 L), 1:1 sodium bicarbonate:water (1.5 L) and again with 1:1 brine:water (1.5 L). The organic phase was dried over magnesium sulfate and concentrated to ca. 1.5 L. To this was added heptane (1.5 L) and this was concentrated to dryness. The resulting solid was triturated with a mixture of methyl t-butyl ether (210 mL) and heptane (220 mL) and the solids were collected by filtration. This material was combined with a smaller scale preparation (28 g) and dried under reduced pressure to give 338 g of the product. ¹H NMR (DMSO-d₆, 400 MHz) δ ppm: 8.68 (d, J=8.8 Hz, 1H), 7.53 (d, J=3.3 Hz, 1H), 7.49 (d, J=8.6 Hz, 1H), 7.32 (dd, J=8.5, 2.6 Hz, 1H), 4.51 (dd, J=8.7, 3.2 Hz, 1H), 4.22 (m, 2H), 4.16 (m, 1H), 3.65 (m, 2H), 3.64 (s, 3H), 3.29 (s, 3H), 1.13 (d, J=6.4 Hz, 3H), 1.08 (s, 9H).

### Step 3. Methyl N-[(2-amino-4-{[2-(methyloxy)ethyl]oxy}phenyl)carbonyl]-O-(1,1-dimethylethyl)-L-threoninate

Methyl O-(1,1-dimethylethyl)-N-[(4-{[2-(methyloxy)ethyl]oxy}-2-nitrophenyl)carbonyl]-L-threoninate (100.3 g, 0.243 mol) was dissolved in methanol (2 L) and the reaction flask was evacuated and flushed 3 times with nitrogen. To this was added palladium on carbon (10%, 10 g) and the mixture was stirred under a hydrogen atmosphere for ca. 4 h. The mixture was filtered and concentrated under reduced pressure. The residue was dissolved in toluene (0.25 L) and concentrated under reduced pressure to give 91.4 g of the product as an amber oil (98%). ¹H NMR (CDCl₃, 400 MHz) δ ppm: 7.38 (d, J=8.7 Hz, 1H), 6.70 (d, J=9 Hz, 1H), 6.31 (dd, J=8.7, 2 Hz, 1H), 6.21 (d, J=2 Hz, 1H), 5.80 (br, 2H), 4.62 (dd, J=9, 1.7 Hz, 1H), 4.27 (m, 1H), 4.09 (m, 2H), 3.72 (m, 2H), 3.71 (s, 3H), 3.43 (s, 3H), 1.22 (d, J=6.1 Hz, 3H), 1.12 (s, 9H).

This procedure was repeated two times starting with 20.5 g and 160 g of the nitro compound to give an additional 162.8 g of product.

### Step 4. N′-(4-Bromo-2,6-dimethylphenyl)-N,N-dimethylimidoformamide

### Method 1

To DMF (21.3 mL, 0.275 mol) in dichloromethane (0.50 L), in reaction flask equipped with a mechanical stirrer, under a nitrogen atmosphere, was added oxalyl chloride (23.3 mL, 0.275 mol) over 1 h. After stirring 10 min, 4-bromo-2,6-dimethylaniline (50.0 g, 0.250 mol) in dichloromethane (0.20 L) was added at such a rate to maintain the reaction temperature between ca. 22 and 27° C. (ca. 1.5 h). The reaction was stirred for ca. 1 h and then quenched by the addition of 2N sodium hydroxide (0.25 L) at such a rate to maintain the reaction temperature between 10 and 15° C. The layers were separated and the organic layer was washed with water (0.20 L), dried over magnesium sulfate and concentrated to dryness. The residue was dissolved in hexanes (0.4 L) and this was concentrated to dryness. The product was pumped on under reduced pressure to give 62.2 g (97.8%) of the product as a dark brown oil. ¹H NMR (DMSO-d₅, 400 MHz) δ ppm: 7.28 (s, 1H), 7.09 (s, 2H), 2.90 (s, 6H), 2.00 (s, 6H).

### Method 2

To a slurry of Vilsmeier reagent (43.2 g, 0.34 mol) in dichloromethane (0.40 L), in a reaction flask equipped with a mechanical stirrer, under a nitrogen atmosphere, was added 4-bromo-2,6-dimethylaniline (61.4 g, 0.31 mol) in dichloromethane (0.20 L) at such a rate to maintain the reaction temperature between ca. 20 and 28° C. (ca. 1.5 h). The reaction was stirred for ca. 1 h and then quenched by the addition of 2N sodium hydroxide (0.35 L) at such a rate to maintain the reaction temperature between 10 and 15° C. The layers were separated and the organic layer was washed with water (0.3 L), dried over magnesium sulfate and concentrated to dryness. The residue was dissolved in hexanes (0.4 L) and this was concentrated to dryness. The product was pumped on under reduced pressure to give 75.7 g (97%) of the product as a dark brown oil. Spectral properties were the same as above.

### Scale up of Method 2

To a slurry of Vilsmeier reagent (211.8 g, 1.66 mol) in dichloromethane (1.97 L), in a jacketed laboratory reactor equipped with a mechanical stirrer, under a nitrogen atmosphere, was added 4-bromo-2,6-dimethylaniline (301.91 g, 1.509 mol) in dichloromethane (0.98 L) at such a rate to maintain the reaction temperature <25° C. (ca. 1.5 h). The reaction was stirred for ca. 1 h and then quenched by the addition of 2N sodium hydroxide (1.72 L) at such a rate to maintain the reaction temperature between 10 and 15° C. The layers were separated and the organic layer was washed with water (1.48 L), dried over sodium sulfate and concentrated to dryness. The residue was dissolved in hexanes (1 L) and this was concentrated to dryness. This was repeated and the product was pumped on under reduced pressure to give 376.82 g (97.8%) of the product as a dark brown oil. Spectral properties were the same as above.

### Step 5. N′-{4-[Cyclopropyl(hydroxy)methyl]-2,6-dimethylphenyl}-N,N-dimethylimidoformamide

N′-(4-bromo-2,6-dimethylphenyl)-N,N-dimethylimidoformamide (489.7 g, 1.92 mol) was dissolved in THF (5 L) in a jacketed laboratory reactor equipped with a mechanical stirrer under a nitrogen atmosphere. The mixture was cooled to −70° C. and n-butyl lithium (2.5 N in hexanes, 1.152 L, 2.88 mol) was added at such a rate to maintain the internal temperature between ca. −65 and −70° C. Once the addition was complete, cyclopropanecarbaldehyde ((201.86 g, 2.88 mol) was added at such a rate to maintain the internal temperature between ca. −50 and −70° C. The reaction was allowed to warm to 0° C. and was quenched by dropwise addition of water (1.96 L) at such a rate that the internal temperature was maintained between 0 and 5° C. The organic solvent was removed under reduced pressure and the remaining water layer and suspended product were transfer to a jacketed laboratory reactor to be used directly in the next step. An aliquot was removed for mass spectral analysis; ES MS m/z 247 (M+H).

### Step 6. (4-Amino-3,5-dimethylphenyl)(cyclopropyl)methanol

To the material from the previous step (N′-{4-[cyclopropyl(hydroxy)methyl]-2,6-dimethylphenyl}-N,N-dimethylimidoformamide) was added a suspension of lithium hydroxide (305 g, 12.73 mol) in water (1.96 L), followed by the addition of ethanolamine (221.48 g, 3.63 mol). The mixture was heated at reflux for ca. 16 h. The reaction was distilled to remove 980 mL of liquid and isopropanol (1.47 L) was added and the mixture was returned to reflux for ca. 5 h. After cooling to RT ethyl acetate (2.5 L) was added and the layers were separated. Both layers were filtered to remove solids and the aqueous layer was back extracted with ethyl acetate (2×1 L). The combined organic layers were washed with water (2×0.5 L), dried over sodium sulfate and concentrated to a volume of ca. 1.4 L. The resulting solids were collected by filtration and were washed with ethyl acetate and hexanes. After drying under reduced pressure 142.47 g of the product was obtained as a white solid. ¹H NMR (DMSO-d₆, 400 MHz) δ ppm: 6.79 (s, 2H), 4.72 (d, J=4.1 Hz, 1H), 4.36 (s, 2H), 3.72 (dd, J=7.2, 4.2 Hz, 1H), 2.06 (s, 6H), 0.91 (m, 1H), 0.12-0.46 (m, 4H).

The mother liquors yielded an additional 37.91 g of product with identical spectral properties. Total yield was 49% for two steps.

### Step 7. [4-(Cyclopropylmethyl)-2,6-dimethylphenyl]amine hydrochloride

To (4-amino-3,5-dimethylphenyl)(cyclopropyl)methanol (169.13 g, 0.884 mol) in dichloromethane (1.68 L) cooled to ca. 15° C. was added triethylsilane (123.4 g, 1.06 mol), followed by trifluoroacetic acid (83.7 g, 0.734 mol) at a rate to maintain the internal reaction temperature between 15 and 16° C. The reaction was stirred for 7 h and then the dichloromethane was distilled off and ethyl acetate was added (1.68 L). This was distilled to a volume of ca. 1.1 L and an additional 280 mL of ethyl acetate was added. With stirring at 20° C., concentrated HCl (73 mL) was added and the mixture was stirred for ca. 15 min. The resulting solids were collected by filtration and washed with 1:1 ethyl acetate:hexanes to give 177.22 g of the product as a tan solid (95%). ¹H NMR (DMSO-d₆, 400 MHz) δ ppm: 9.13-9.91 (brs, 2H), 7.01 (s, 2H), 2.41 (d, J=6.9 Hz, 2H), 2.34 (s, 6H), 0.92 (m, 1H), 0.47 (m, 2H), 0.15 (m, 2H).

### Step 8. 5-(Cyclopropylmethyl)-2-isocyanato-1,3-dimethylbenzene

To a solution of triphosgene (81.3 g, 0.274 mol) in dichlorormethane (0.855 L) was added a mixture of [4-(cyclopropylmethyl)-2,6-dimethylphenyl]amine hydrochloride (115 g, 0.543 mol) and DIEA (176.7 g, 1.37 mol) in dichloromethane (0.3 L) maintaining the temperature less than 25° C. The reaction was stirred ca. 16 h and heptane (1.0 L) was added. The mixture was washed with water (2 times 0.30 L). The aqueous phases were back extracted with heptane (2 time 0.10 L) and the combined organics were dried over magnesium sulfate. Concentration under reduced pressure gave 98.95 g of the product as an oil (91% yield). ¹H NMR (CDCl₃, 400 MHz) ppm: 6.92 (s, 2H), 2.41 (d, J=6.8 Hz, 2H), 2.30 (s, 6H), 0.93 (m, 1H), 0.50 (m, 2H), 0.16 (m, 2H).

### Step 9. Methyl N-[(2-[({[4-({[4-(cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threoninate

Methyl N-[(2-amino-4-{[2-(methyloxy)ethyl]oxy}phenyl)carbonyl]-O-(1,1-dimethylethyl)-L-threoninate (144 g, 0.377 mol) and 5-(cyclopropylmethyl)-2-isocyanato-1,3-dimethylbenzene (98.9 g, 0.491 mol) were combined in pyridine (1.0 L) and the mixture was stirred at RT of ca. 16 h. The solvent was removed under reduced pressure and the residue was dissolved in methyl t-butyl ether (1 L). This was washed with 0.1 N HCl (4 times 0.5 L). The solvent was removed under reduced pressure and the residue was chromatographed on silica gel (1.5 Kg), eluting with ethyl acetate/hexane (gradient), to give 181 g of the product as a foam. ¹H NMR (DMSO-d₆, 400 MHz) 8 ppm: 10.42 (brs, 1H), 8.70 (brs, 1H), 8.01 (brs, 1H), 7.97 (d, J=2.2 Hz, 1H), 7.66 (d, J=8.1 Hz, 1H), 6.92 (s, 2H), 6.57 (dd, J=8.8, 2.5 Hz, 1H), 4.45 (m, 1H), 4.16 (m, 1H), 4.05 (m, 2H), 3.64 (s, 3H), 3.62 (m, 2H), 3.27 (s, 3H), 2.39 (d, J=6.8 Hz, 2H), 2.11 (s, 6H), 1.14 (m, 3H),

### Step 10. N-[(2-[({[4-(Cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threonine

To methyl N-[(2-[({[4-(cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threoninate (179 g, 0.306 mmol) in THF (1 L) was added lithium hydroxide mono-hydrate (38.5 g, 0.92 mol) in water (0.33 L) drop wise over ca. 30 min. The mixture was stirred at RT for 2 days, and then cooled in an ice bath and the pH was adjusted to ca. 1 by the addition of 6 N HCl. To this was added ethyl acetate (1.5 L) and the mixture was stirred at 10° C. for ca. 30 min. The layers were separated and the aqueous phase was extracted with ethyl acetate (0.2 L). The combined organics were dried over magnesium sulfate, filtered and concentrated under reduced pressure to give 180 g of the product, which contained some ethyl acetate and THF. This material (combined with a smaller 9 g batch) was crystallized from acetonitrile (1.7 L) and water (1.36 L) to give, after drying under reduced pressure at 50° C., 125.5 g of the product as a white solid. ¹H NMR (DMSO-d₆, 400 MHz) 5 ppm: 12.78 (brs, 1H), 10.52 (brs, 1H), 8.68 (brs, 1H), 7.98 (d, J=2.2 Hz, 1H), 7.75 (brs, 1H), 7.63 (d, J=7.9 Hz, 1H), 6.90 (s, 2H), 6.58 (dd, J=8.8, 2.4 Hz, 1H), 4.37 (m, 1H), 4.16 (m, 1H), 4.06 (m, 2H), 3.62 (m, 2H), 3.27 (s, 3H), 2.39 (d, J=6.8 Hz, 2H), 2.11 (s, 6H), 1.11 (m, 3H), 1.11 (s, 9H), 0.92 (m, 1H), 0.43 (m, 2H), 0.15 (m, 2H). Anal. calculated for C₃₁H₄₃N₃O₇: C, 65.36; H, 7.61; N, 7.38. Found: C, 65.59; H, 7.59; N, 7.41.

### Example 3

### Preparation of the Potassium Salt of the Compound of Formula IA

### Potassium N-[(2-[({[4-(cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threoninate

To N-[(2-[({[4-(cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threonine (1.0 g, 1.76 mmol) in acetonitrile (100 mL) is added potassium t-butoxide (1.0 M in THF, 1.76 mL). The mixture is stirred for ca. 15 min and the solvent is removed under reduced pressure to give the product.

### Biological Protocols

The utility of the compounds of Formula I, a salt, solvate, or physiologically functional derivative thereof, in the treatment or prevention of diseases (such as detailed herein) in animals, particularly mammals (e.g., humans) may be demonstrated by the activity in conventional assays known to one of ordinary skill in the relevant art, including the in vitro and in vivo assays described below.

The purified glycogen phosphorylase (GP) enzyme, wherein glycogen phosphorylase is in the activated “a” state, referred to as human liver glycogen phosphorylase a (HLGPa), can be obtained according to the following procedures.

### Appropriate Cloning and Expression of Human Liver Glycogen Phosphorylase:

Human liver glycogen phosphorylase cDNA was amplified by polymerase chain reaction (PCR) from a commercially available human liver cDNA library (BD Biosciences). The cDNA was amplified as 2 overlapping fragments using the primers 5′GGCGAAGCCCCTGACAGACCAGGAGAAG3′ with 5′CGATGTCTGAGTGGATTTTAGCCACGCC3′ and 5′GGATATAGAAGAGTTAGAAGAAATTG3′ with 5′GGAAGCTTATCAATTTCCATTGACTTTGTTAGATTCATTGG3′. PCR conditions were 94° C. 1 min., 55° C. 1 min., 72° C. 2 min. for 40 cycles using the enzyme Pfu Turbo (Stratagene), 0.5% DMSO, 250 uM each nucleotide triphosphate, and 0.4 uM each primer plus the buffer recommended by the polymerase manufacturer. Each PCR fragment was molecularly cloned and the DNA sequence of each insert was determined. The 2 DNA fragments of the glycogen phosphorylase cDNA were then joined together in a bacterial expression plasmid, pTXK1007LTev (GlaxoSmithKline), creating a full-length cDNA fused at the 5′ end to codons for methionine-glycine-alanine-histidine-histidine-histidine-histidine-histidine-histidine-glycine-glycine-glutamate-asparagine-leucine-tyrosine-phenylalanine-glutamine-glycine-glycine-. The protein product would have a 6× histidine tag followed by a Tev protease cleavage site. The DNA sequence of both strands of the cDNA in pTXK1007LTev was determined.

### Purification of Human Liver Glycogen Phosphorylase:

The frozen cell paste (100 g) was thawed and suspended in 1200 ml of 50 mM Tris, 100 mM NaCl, 15 mM imidazole, pH 8.0. The cells were disrupted gently with a Polytron (Brinkman, PT10-35), and passed twice through an AVP homogenizer. The E. coli cell lysates were clarified by centrifugation at 27,500×g for 45 minutes and filtered through a 0.8 micron filter. The solution was applied to a 21 ml Ni-NTA Superflow (Qiagen) column (ID 26 mm×H 4.0 cm) pre-equilibrated with 50 mM Tris, 100 mM NaCl, and 15 mM imidazole, pH 8.0. The column was washed with equilibration buffer until the A280 returned to baseline. The weakly bound proteins were eluted from the column with 10 bed column volumes of 50 mM imidazole in the same buffer. The glycogen phosphorylase was eluted with steps of 100 mM and 250 mM imidazole. Both the 100 mM and 250 mM fractions were pooled and then diluted 5 fold with 50 mM Tris, pH 8.0 buffer. This solution was loaded on a 21 ml Q fast flow column (Amersham Pharmacia Biotech AB, ID 2.6 cm×H 4.0 cm) pre-equilibrated with 50 mM Tris, pH 8.0. Glycogen phosphorylase was eluted with a continuous gradient from 0-30% of 1M NaCl in 50 mM Tris, pH 8.0 (buffer B). Fractions of purified glycogen phosphorylase between 15% and 20% buffer B were pooled, aliquoted into microfuge tubes, and stored at −80° C. The purified fraction formed a single ˜100 kd band on a SDS-PAGE gel.

### Activation of Human Liver Glycogen Phosphorylase:

The activation of human liver glycogen phosphorylase (i.e., conversion of the inactive HLGPb form to the activated HLGPa form) was achieved by phosphorylating HLGPb with immobilized phosphorylase kinase.

10 mg of phosphorylase kinase (Sigma, P-2014) was dissolved in 2.5 ml of 100 mM HEPES, 80 mM CaCl2 (pH 7.4) and gently mixed with 1 ml of Affi-Gel (Active Ester Agarose, BioRad #153-6099) beads previously equilibrated in the same buffer. The mixture was rocked 4 hours at 4° C. The beads were washed once with the same buffer and blocked for 1 hour at room temperature with a solution of 50 mM HEPES, 1M glycine methyl ester, pH 8.0. The beads were then washed with 50 mM HEPES, 1 mM β-mercaptoethanol, pH 7.4 and stored at 4° C.

Frozen purified glycogen phosphorylase (HLGPb) was thawed in at 4° C. then dialyzed overnight into 50 mM HEPES, 100 mM NaCl, pH 7.4. 15 mg of the dialyzed HLGPb, 3 mM ATP and 5 mM MgCl2 was incubated with 500 ul of the prepared Affi-Gel immobilized phosphorylase kinase beads equilibrated with 50 mM HEPES, 100 mM NaCl, pH 7.4. The degree of phosphorylation was monitored by following the increase in activity at 10 minute intervals using the assay system outlined below. Briefly, the assay contained 0.1 uM human liver glycogen phosphorylase, 50 mM HEPES, 100 mM KCl, 2.5 mM EGTA. MgCl₂, 3.5 mM KH₂PO₄, 0.5 mM DTT, 0.4 mg/mL glycogen, 7.5 mM Glucose, 0.50 mM β-nicotinamide adenine dinucleotide (β-NAD), 3 U/mL phosphoglucomutase, and 5 U/mL glucose-6-phosphate dehydrogenase. Activity was monitored by following the reduction of NAD⁺ at 340 nm. The reaction was stopped by removal of the beads from the mixture when no further increase in activity was observed (30-60 minutes). Phosphorylation was further confirmed by analysis of the sample by mass spectroscopy. The supernatant containing the activated sample was dialyzed in 50 mM HEPES, 100 mM NaCl, pH 7.4 overnight. The final sample was mixed with an equal volume of glycerol, aliquoted into microfuge tubes and stored at −20° C.

### Human Liver Glycogen Phosphorylase a Enzymatic Activity Assay:

An enzymatic assay was developed to measure the response of the activated form of glycogen phosphorylase (HLGPa) to small molecule (<1000 Da.) compounds. The assay was configured to monitor the pharmacologically relevant glycogenolytic reaction by coupling the production of glucose-1-phosphate from glycogen and inorganic phosphate to phosphoglucomutase, glucose-6-phosphate dehydrogenase, NADH oxidase and horseradish peroxidase to produce the fluorescent product resorufin. The concentrations of the reagent components were as follows: 15 nM human liver glycogen phosphorylase a, 1 mg/mL glycogen, 5 mM K₂HPO₄, 40 U/mL phosphoglucomutase (Sigma), 20 U/mL glucose-6-phosphate dehydrogenase (Sigma), 200 nM Thermus thermophilus NADH oxidase (prepared as described in Park, H. J.; Kreutzer, R.; Reiser, C. O. A.; Sprinzl, M. Eur. J. Biochem. 1992, 205, 875-879), 2 U/mL horseradish peroxidase (Sigma), 30 uM FAD, 250 uM NAD⁺, 50 uM amplex red, +/−10 mM glucose. The base assay buffer used was 50 mM HEPES, 100 mM NaCl, pH 7.6. To aid in the identification of glucose-sensitive inhibitors of glycogen phosphorylase, the assay was performed with and without 10 mM glucose. In order to scrub the assay of contaminating components that may contribute to non-HLGPa specific resorufin production, the reagents were prepared as two 2× concentrated cocktails. A solution of catalase-coated agarose beads was prepared in the base assay buffer. The first cocktail (cocktail #1) consisted of Thermus thermophilus NADH oxidase, NAD⁺, glycogen, phosphoglucomutase, glucose-6-phosphate dehydrogenase, K₂HPO₄, FAD, and 50 U/mL catalase-coated agarose beads +/−10 mM glucose. Amplex red was added to this solution after incubation at 25° C. for 30 minutes and the catalase-coated agarose beads were removed by centrifugation and retention of supernatant. The second cocktail (cocktail #2) contained human liver glycogen phosphorylase-a and horseradish peroxidase +/−10 mM glucose. The assays were performed with preincubation of compounds of this invention with cocktail #2 for 15 minutes, followed by the addition of cocktail #1 to initiate the reaction. The assays were performed in 96 (black % volume Costar #3694) or 384-well microtiter plates (small volume black Greiner). The change in fluorescence due to product formation was measured on a fluorescence plate reader (Molecular Devices SpectraMax M2) with excitation at 560 nm and emission at 590 nm. Activity of example compound 1 is shown in Table 1 below.

TABLE 1
Activity of the compound in human liver glycogen phosphorylase a enzymatic assay.

| | | | ESMS + | IC50 |
|---|---|---|---|---|
| Ex # | Structure | Chemical Name | m/z | (uM) |
| 1 | 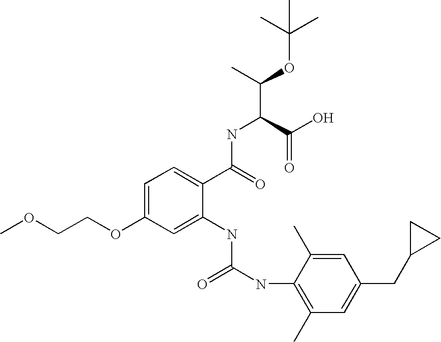 | N-[(2-[({[4- (cyclopropylmethyl)-2,6- dimethylphenyl]amino} carbonyl)amino]-4-{[2- (methyloxy)ethyl]oxy} phenyl)carbonyl]-O-(1,1- dimethylethyl)-L-threonine | 570 (M + H) | 0.004 |

### In Vivo Glucagon Challenge Model:

Jugular vein cannulated male CD rats (220-260 g) (Charles Rivers, Raleigh, N.C.) were received 1-2 days after cannulation, housed individually on Alpha-dri™ bedding (Shepherd Specialty Papers, Inc., Kalamazoo, Mich.) with free access to food (Lab Diet 5001, PMI Nutrition International, Brentwood, Mo.) and water and maintained on a 12 h light/dark cycle at 21° C. and 50% relative humidity for 3-4 days prior to the glucagon challenge studies. On the day of the study, the rats were sorted by body weight into treatment groups (N=4-5) and housed individually in shoe box cages with clean Alpha-dri bedding. The cannula lines were opened by removal of 0.2 ml blood and flushed with 0.2 ml sterile saline. After a one hour acclimation, blood samples were collected to determine basal glucose and the rats were orally dosed with vehicle (5% DMSO: 30% Solutol HS15: 20% PEG400: 45% 25 mM N-methylglucamine) or drug (5 ml/kg). Two hr after drug dosing, a time zero blood sample (0.4 ml) was collected for determination of glucose and the rats were dosed through the jugular vein with Sandostatin, 0.5 mg/kg, (Novartis Pharmaceuticals Corp., East Hanover, N.J.) and glucagon, 10 ug/kg (Bedford Laboratories, Bedford, Ohio). Blood samples were collected after 10 and 20 min for glucose determination. Whole blood was placed in a Terumo Capiject blood collection tube (Terumo Medical Corp., Elkton, Md.), allowed to sit at room temperature for 20-30 minutes and then centrifuged (3,000×G) to obtain serum. Serum levels of glucose were determined using an Olympus AU640™ clinical chemistry immuno-analyzer (Olympus America Inc., Melville, N.Y.). The % reduction (% R) of the vehicle glucose AUC was calculated for each drug treatment using the formula % reduction=100*1−(AUC drug/AUC vehicle), where AUG was calculated from serum glucose values using the equation AUG=(T0+T10)/2*10+(T10+T20)/2*10−(T0*20). Activity of example compound 1 is shown in Table 2 below.

TABLE 2
Activity of the compound in the in vivo glucagon challenge model.

| Dose of N-[(2-[({[4-(Cyclopropylmethyl)- | |
|---|---|
| 2,6-dimethylphenyl]amino}carbonyl)amino]- | |
| 4-{[2-(methyloxy)ethyl]oxy}phenyl)carbonyl]- | |
| O-(1,1-dimethylethyl)-L-threonine (example | |
| compound 1) (mg/kg) | % R |
| 0.1 | 4 |
| 0.5 | 36 |
| 2 | 48 |
| 5 | 76 |

What is claimed is:

1-15. (canceled)

16. A compound of Formula I 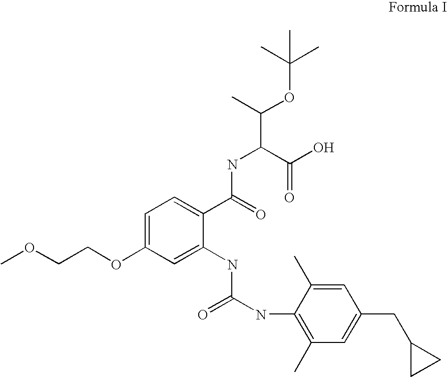
or a salt thereof.

17. The compound of claim 16 wherein the stereochemistry is that shown in Formula IA 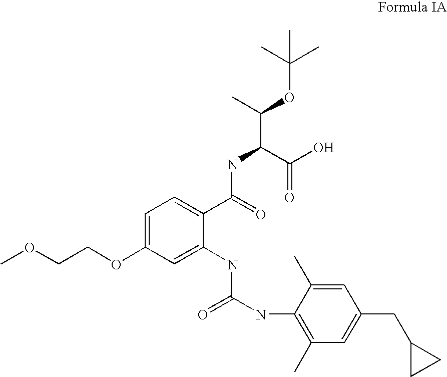
or a salt thereof.

18. A pharmaceutical composition comprising a compound of claim 16 or a salt thereof.

19. A pharmaceutical composition comprising a compound of claim 17 or a salt thereof.

20. A pharmaceutical composition comprising a compound of claim 16 or a salt thereof and one or more excipients.

21. A pharmaceutical composition comprising a compound of claim 17 or a salt thereof and one or more excipients.

22. The pharmaceutical composition of claim 20 in the form of a tablet or capsule.

23. The pharmaceutical composition of claim 21 in the form of a tablet or capsule.

24. A method of treatment comprising the administering to a mammal a pharmaceutical composition comprising a compound of claim 20, or a pharmaceutically acceptable salt thereof and at least one excipient, wherein said treatment is for a disease or condition selected from the group consisting of diabetes and conditions associated with diabetes.

25. The method of treatment of claim 24 wherein said mammal is a human.

26. A method of treatment comprising the administering to a mammal a pharmaceutical composition comprising a compound of claim 21, or a pharmaceutically acceptable salt thereof and at least one excipient, wherein said treatment is for a disease or condition selected from the group consisting of diabetes and conditions associated with diabetes.

27. The method of treatment of claim 26 wherein said mammal is a human.

28. The method of claim 24 wherein said conditions associated with diabetes are selected from the group consisting of obesity, syndrome X, insulin resistance, diabetic nephropathy, diabetic neuropathy, diabetic retinopathy, hyperglycemia, hypercholesterolemia, hyperinsulinemia, hyperlipidemia, cardiovascular disease, stroke, atherosclerosis, lipoprotein disorders, hypertension, tissue ischemia, myocardial ischemia, and depression.

29. The method of claim 26 wherein said conditions associated with diabetes are selected from the group consisting of obesity, syndrome X, insulin resistance, diabetic nephropathy, diabetic neuropathy, diabetic retinopathy, hyperglycemia, hypercholesterolemia, hyperinsulinemia, hyperlipidemia, cardiovascular disease, stroke, atherosclerosis, lipoprotein disorders, hypertension, tissue ischemia, myocardial ischemia, and depression.

30. The method of claim 24 wherein said treatment is for diabetes.

31. The method of claim 26 wherein said treatment is for diabetes.

32. A process for preparing a compound of claim 16 salt thereof comprising the steps of:
a. conversion of 4-fluoro-2-nitrobenzoic acid to 4-{[2-(methyloxy)ethyl]oxy}-2-nitrobenzoic acid;
b. conversion of 4-{[2-(methyloxy)ethyl]oxy}-2-nitrobenzoic acid to methyl O-(1,1-dimethylethyl)-N-[(4-{[2-(methyloxy)ethyl]oxy}-2-nitrophenyl)carbonyl]-L-threoninate;
c. conversion of methyl O-(1,1-dimethylethyl)-N-[(4-{[2-(methyloxy)ethyl]oxy}-2-nitrophenyl)carbonyl]-L-threoninate to methyl N-[(2-amino-4-{[2-(methyloxy)ethyl]oxy}phenyl)carbonyl]-O-(1,1-dimethylethyl)-L-threoninate;
d. conversion of 4-bromo-2,6-dimethylaniline to N′-(4-bromo-2,6-dimethylphenyl)-N,N-dimethylimidoformamide;
e. conversion of N′-(4-bromo-2,6-dimethylphenyl)-N,N-dimethylimidoformamide to N′-{4-[cyclopropyl(hydroxy)methyl]-2,6-dimethylphenyl}-N,N-dimethylimidoformamide;
f. conversion of N′-{4-[cyclopropyl(hydroxy)methyl]-2,6-dimethylphenyl}-N,N-dimethylimidoformamide to (4-amino-3,5-dimethylphenyl)(cyclopropyl)methanol;
g. conversion of (4-amino-3,5-dimethylphenyl)(cyclopropyl)methanol to [4-(cyclopropylmethyl)-2,6-dimethylphenyl]amine hydrochloride;
h. conversion of [4-(cyclopropylmethyl)-2,6-dimethylphenyl]amine hydrochloride to 5-(cyclopropylmethyl)-2-isocyanato-1,3-dimethylbenzene;
i. conversion of methyl N-[(2-amino-4-{[2-(methyloxy)ethyl]oxy}phenyl)carbonyl]-O-(1,1-dimethylethyl)-L-threoninate and 5-(cyclopropylmethyl)-2-isocyanato-1,3-dimethylbenzene to methyl N-[(2-[({[4-(cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threoninate; and
j. conversion of methyl N-[(2-[({[4-(cyclopropylmethyl)-2,6-dimethylphenyl]amino}carbonyl)amino]-4-{[2-(methyloxy)ethyl]oxy}phenyl)carbonyl]-O-(1,1-dimethylethyl)-L-threoninate to N-[(2-[({[4-(cyclopropylmethyl)-2,6-dimethylphenyl]amino}carbonyl)amino]-4-{[2-(methyloxy)ethyl]oxy}phenyl) carbonyl]-O-(1,1-dimethylethyl)-L-threonine.


SEQUENCE LISTING (4 sequences)

SEQ ID NO: 1 - DNA, 28 bp, Homo Sapien
ggcgaagcccctgacagaccaggagaag

SEQ ID NO: 2 - DNA, 28 bp, Homo Sapien
cgatgtctgagtggattttagccacgcc

SEQ ID NO: 3 - DNA, 26 bp, Homo Sapien
ggatatagaagagttagaagaaattg

SEQ ID NO: 4 - DNA, 41 bp, Homo Sapien
ggaagcttatcaatttccattgactttgttagattcattgg